(19) United States
(12) Patent Application Publication
(10) Pub. No.: US 2008/0108948 A1
(43) Pub. Date: May 8, 2008

(54) METHOD AND APPARATUS FOR SECURING A SURGICAL DRAIN

(76) Inventor: PAMELA L BEAVER, LOS ANGELES, CA (US)

Correspondence Address: THE HECKER LAW GROUP, 1925 CENTURY PARK EAST, SUITE 2300, LOS ANGELES, CA, 90067, US

(21) Appl. No.: 11/557,418

(22) Filed: Nov. 7, 2006

(51) Int. Cl. A61M 27/00

(52) U.S. Cl. 604/179

(57) ABSTRACT

An apparatus for securing one or more drainage systems, such as one or more surgical drains associated with post-operative recovery, is presented. The apparatus includes a base fabric comprising material which acts as the hooks of a hook-and-loop fastener on its rear side for securing the drain system to a supporting structure, such as post operative dressing. The front side of the base comprises substantially of fabric acting as the loops of a hook-and-loop fastener for securing the reservoir bulb and extension tube of a surgical drain system. A transparent netting type fabric which includes a hooks type fabric provides removable coupling of the reservoir bulb to the front side of the base. A flexible strap which is configured as a hooks fabric on one side and loops fabric on the other side provides removable coupling of the extension tube to the front side of the base.

### FIELD OF INVENTION

This invention relates to the field of medical devices, and more specifically, to securing of post-operative drainage systems.

### BACKGROUND OF INVENTION

Treatment of some health conditions or anomalies (e.g. diseases or injuries) may require surgery, i.e., treatment by operative manual and instrumental procedures. And surgery generally requires a substantial amount of healing time. Of course, the amount of healing time and associated care depends on the type of surgery performed. For instance, major surgeries, such as plastic surgery and orthopedic surgery, may require substantial healing time and special care, compared to minor surgeries.

Plastic surgery, especially, requires special care because of the nature of the desired outcome, because plastic surgery is a treatment which is performed for functional or aesthetic reasons. Plastic surgery includes both cosmetic and reconstructive surgery. Generally, cosmetic surgery is used to reshape a person's body in order to improve appearance and self-esteem. Cosmetic surgery procedures include: Abdominoplasty (i.e. Tummy Tuck), Breast Enlargement, Breast Lift, Chemical Peel, Collagen/Fat Injections, Dermabrasion, Ear Surgery, Eyelid Surgery, Facelift, Facial Implants, Forehead Lift, Hair Replacement Surgery, Laser Facial Resurfacing, Liposuction, Male Breast Reduction, Nose Surgery, etc.

Reconstructive surgery is used to correct abnormal structures of the body, caused by congenital defects, developmental abnormalities, trauma, infection, tumors or disease. It is generally performed to improve functions, but may also be done to approximate a normal appearance. Reconstructive Procedures include: Breast Reconstruction, Breast Reduction, Cleft Lip and Palate, Hand Surgery, Male Breast Reduction, Scar Revision, Skin Cancer, Skin Resurfacing, Tissue Expansion.

Irrespective of the specialty area, surgery can be a traumatic event for the patient. Thus, post operative recovery procedures are geared towards uneventful and fast healing. Some surgical procedures, e.g. plastic surgery, may require that fluid be actively drained from the surgical site to aid healing and obtain the desired result.

For instance, it is common practice in some cosmetic or reconstructive surgical procedures to install surgical drainage systems to aid post-operative recovery. A surgical drain is a tube inserted into the surgical site to remove pus, blood or other fluids from the wound. Drains inserted after surgery help the wound heal faster and assist in preventing infection. Drains may be hooked to wall suction, a portable suction device, or they may be left to drain by gravity. A surgical drain can either be open or closed. Closed drains use vacuum bottles (or bulbs) and plastic conduits to draw fluid away from the surgical site by producing a negative pressure.

Surgical drains in use today include: Jackson Pratt, Wound Vac System, Davol, chest tube, etc. The Jackson Pratt drain consists of a tube connected to a see-through collection bulb or reservoir. The bulb has a drainage port which can be opened to remove fluid or air so that the bulb can be squeezed to create suction. The drain is usually placed below the area of the wound.

FIG. 1 is an illustration of a bulb type (Jackson Pratt) drain device employed in post-operative recovery procedures. As illustrated, drain device 100 comprises drain tube 106, coupled to a bulb type drain reservoir 102 through plastic extension tube 104. Drain tube 106 is generally available in either a flat or a round configuration, and in various sizes. In practice, the drain tube is placed in the vicinity of the surgical site to aid in fluid removal.

FIGS. 2 and 3 are illustrations of the placement of a bulb type drain device on a patient with tummy tuck surgery. As illustrated, the drain tube 106 is placed in the vicinity of the surgical site (see approximate placement in FIG. 2) in patient 200, but under the patient's skin during the tummy tuck surgery (not shown). Subsequently, after the operation, the skin is placed over the drains and the surgical site is closed such that only the extension tube (i.e. 104) and the drain reservoir (i.e. 102) are exposed, as illustrated in patient 300 of FIG. 3.

Usually, a type of dressing, e.g. an ACE Bandage or surgical tape, is used to cover the surgical site (e.g. 310) to prevent infection. And, the drain reservoir (i.e. 102) is either taped to the dressing (see FIG. 4) or left hanging (as shown in FIG. 3), i.e., in position to collect fluid (e.g. 305) through vacuum process, gravity, etc.

FIG. 4 is an illustration of the placement of a bulb type drain device on a patient 400 after breast surgery. As illustrated, the drain (not shown) is inserted in the surgical site (i.e. breast) and the surgical site is covered with dressing 410 (e.g. ACE bandage, surgical tape, etc) so that the extension tube, i.e. 406, extends from below the dressing to the drain reservoir 402. In this illustration, the extension tube is secured to the dressing with adhesive tape 404 and the reservoir bulb is left hanging. However, some surgeons may also secure the drain reservoir (e.g. bulb) with adhesive tape 404.

The problem is that the adhesive (i.e. securing) tape does not provide enough binding to keep the reservoir in place. Thus, a recurrent problem is that the drains and reservoirs dislodge from the securing tape thereby reducing or eliminating the effectiveness of the drainage system. And without a secure drain and reservoir system, excess fluid sometimes accumulates in the surgical site thus delaying the healing process.

### SUMMARY OF THE INVENTION

The invention comprises a method and apparatus for securing a surgical drain system. The apparatus and methods of the present invention provide highly configurable means for securing surgical drainage systems to patients during post-operative recovery.

In one or more embodiment, the apparatus comprises a base fabric made of material which acts as the hooks of a hook-and-loop fastener on its rear side for securing the drain system to a supporting structure, such as post operative dressing. The front side of the base fabric comprises substantially of material acting as the loops of a hook-and-loop fastener for securing the reservoir bulb and extension tube of the surgical drain system.

In one embodiment, a sock comprising a transparent netting type fabric is partially configured with a hooks type fabric to provide removable coupling of the reservoir bulb to the loops material on the front side of the base. The sock is further configured with an elastic top for temporarily securing the reservoir bulb inside the sock.

In one embodiment, one or more flexible straps are used to secure extension tubes. Each flexible strap is configured as a hooks fabric on one side and loops fabric on the other side to provide removable coupling of the extension tube to the front side of the base. When securing an extension tube, the strap is rolled around the extension tube such that its hooks fabric is on the outside to provide removable coupling to the front side of the base, which comprises loops fabric.

Further objects, features, and advantages of the present invention over the prior art will become apparent from the detailed description of the drawings which follows, when considered with the attached figures.

### BRIEF DESCRIPTION OF THE DRAWINGS

FIG. 1 is an illustration of a bulb type (Jackson Pratt) drain device employed in post-operative recovery procedures.

FIG. 2 is an illustration of the location of a bulb type drain device on a patient with tummy tuck surgery.

FIG. 3 is an illustration of a patient with a bulb type drain device after tummy tuck surgery.

FIG. 4 is an illustration of a patient with a bulb type drain device after breast surgery.

FIG. 5 is an illustration of the drain securing apparatus, in accordance with an embodiment of the present invention.

FIG. 6 is an exploded view of the drain securing apparatus, in accordance with an embodiment of the present invention.

FIG. 7 is an illustration of the base of the drain securing apparatus, in accordance with an embodiment of the present invention.

FIG. 8 is an illustration of a sock securing a drain reservoir to the base of the drain apparatus, in accordance with an embodiment of the present invention.

FIG. 9 is an illustration of a strap for securing an extension tube to the base of the drain apparatus, in accordance with an embodiment of the present invention.

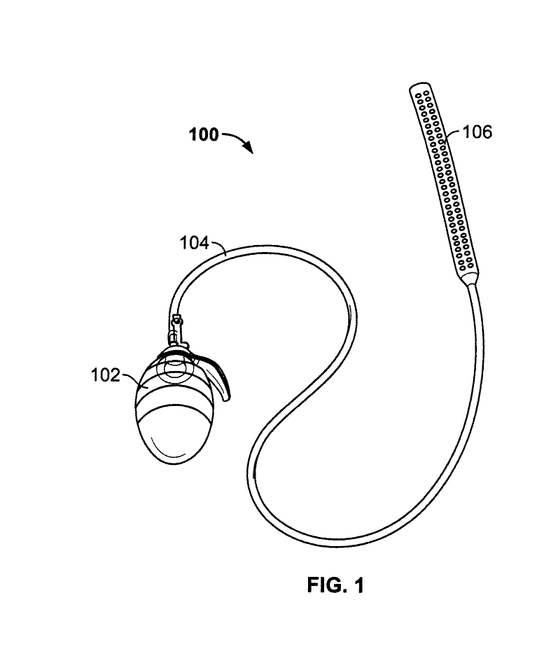

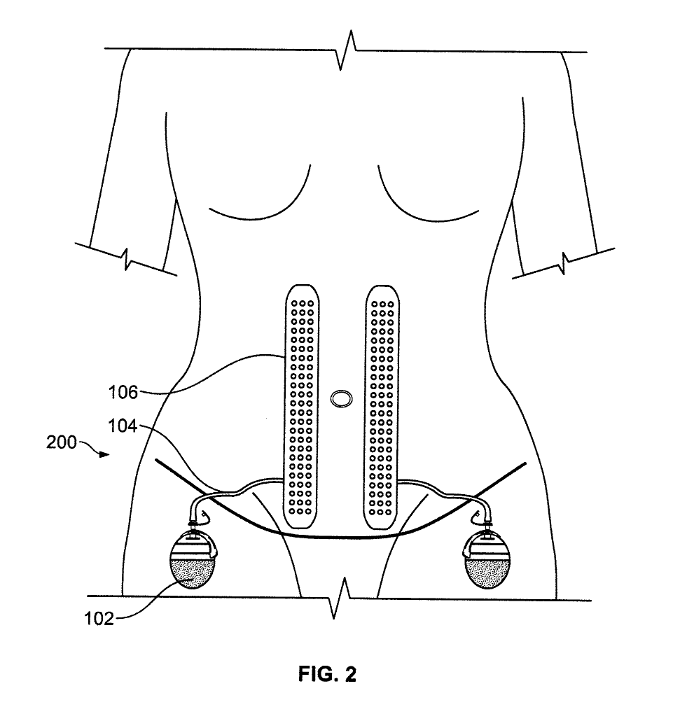

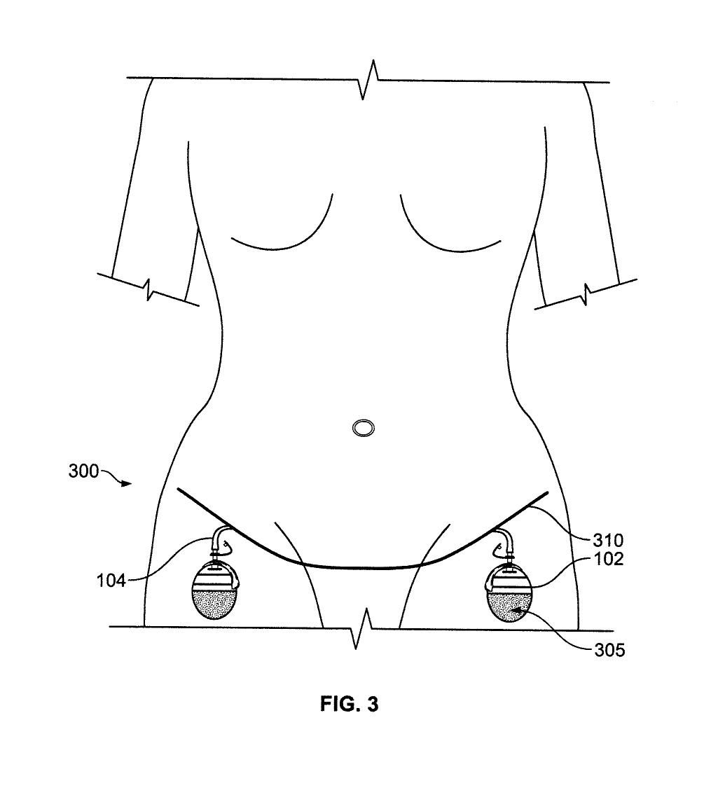

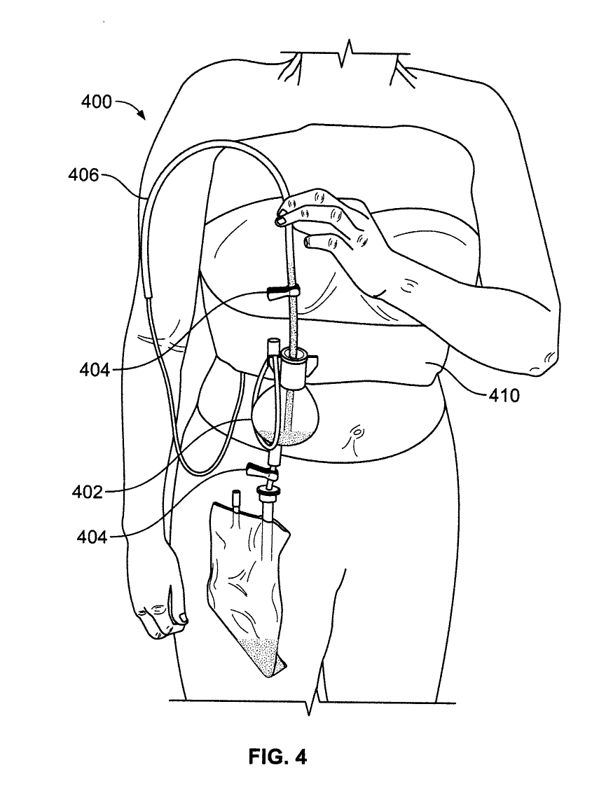

### DETAILED DESCRIPTION OF THE INVENTION

The invention discloses a method and apparatus for securing a surgical drainage system. In the following description, numerous specific details are set forth to provide a more thorough description of the present invention. It will be apparent, however, to one skilled in the art, that the present invention may be practiced without these specific details. In other instances, well known features have not been described in detail so as not to obscure the present invention.

In general, the invention comprises an apparatus for securing one or more drainage systems, such as one or more surgical drains associated with post-operative recovery. In one embodiment, the apparatus includes a base for securing the drain system to a supporting structure, such as post-operative dressing. The apparatus also includes one or more socks which are couplable to the base and configured to support drain reservoirs. Each sock is configured to be removably couplable to any part of the base.

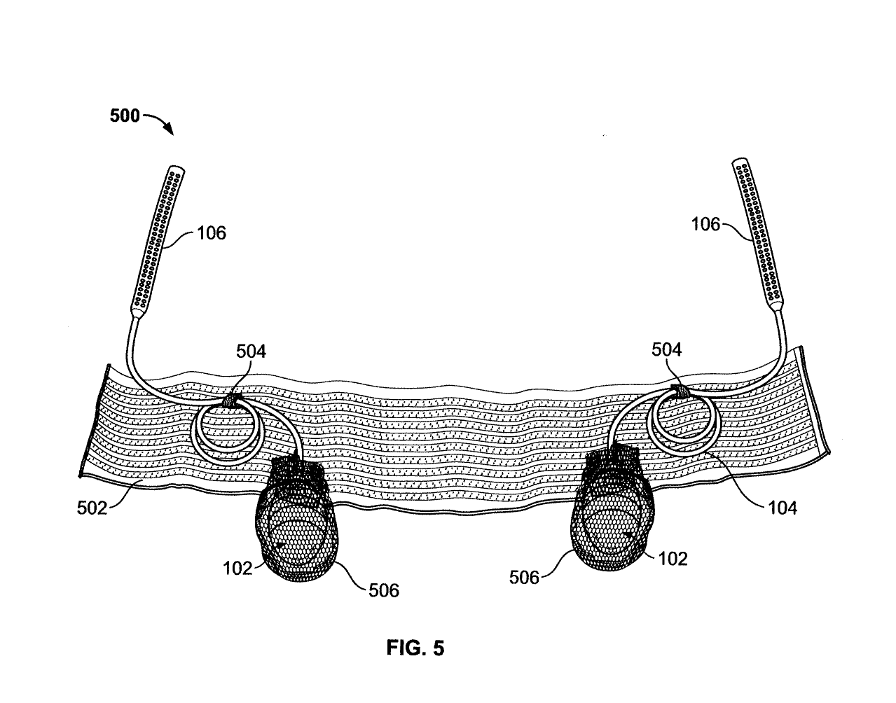

The apparatus also includes one or more straps couplable to the base and configured to secure the extension tube (see FIG. 5). Each strap is also configured to be removably couplable to any part of the base structure.

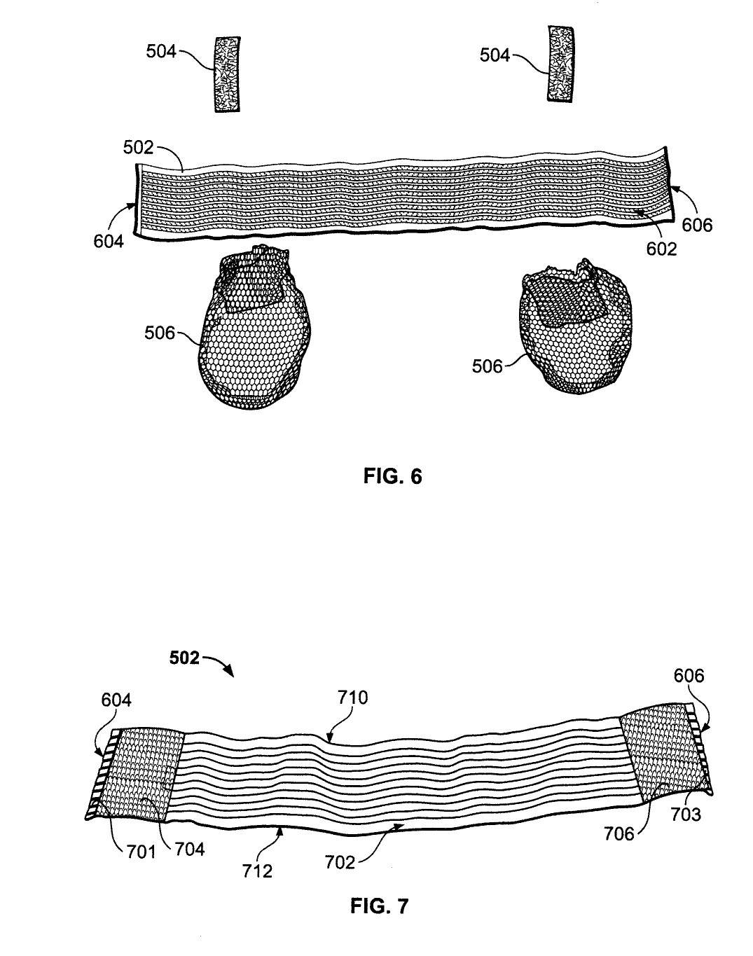

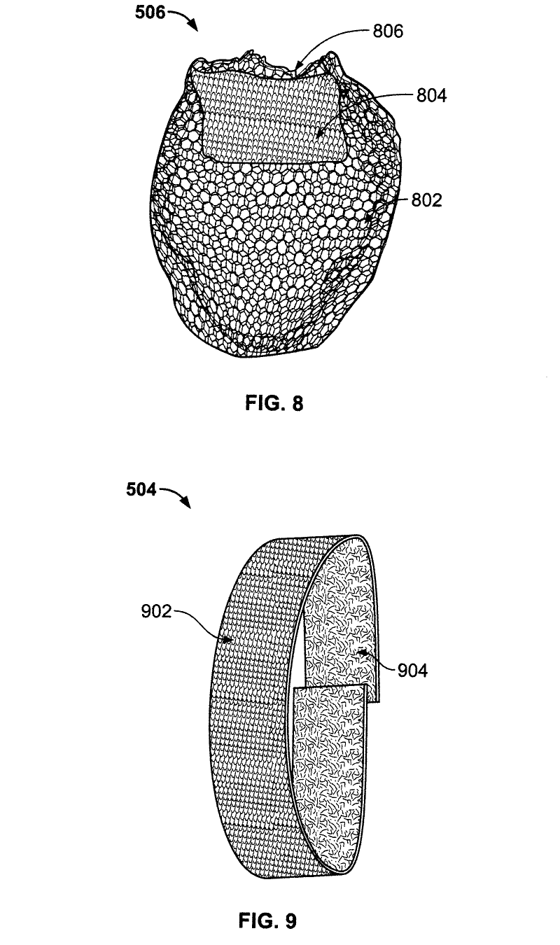

One embodiment of a drainage apparatus 500 of the invention will now be described in more detail using FIGS. 5 to 9. Referring first to FIG. 6, in one embodiment, the drainage apparatus includes a base 502. The base 502 may have a variety of configurations. In one embodiment, the base 502 is a piece of flexible, cloth-like fabric which is generally planar when in its fully open configuration.

As illustrated, the base 502 is generally rectangular in shape. The base 502 has a front side or face 602 and an opposing rear side or face 702. The base 502 has a left side edge 604 and a right side edge 606. The left side and right side edges (i.e. 604 and 606) are located at opposing portions of the base 502, and extend generally parallel to one another when the base is in its fully extended position. In one embodiment, rectangular base 502 is eighteen (18) inches long when full extended. However, the shape and size of base 502 may vary to suit the application. For example, the shape may be configured round, square, etc.

In one embodiment, the front side or face 602 of base 502 comprises a fabric material having hairy fibers which is capable of acting as the loop side of a “hook-and-loop” fastener, i.e. VELCRO®.

On the rear side or face 702 of base 502 is fixably coupled one or more pieces of fabric which is covered with tiny plastic hooks capable of acting as the hook side of a “hook and loop” fastener. The hook fabric may be configured into any shape. For instance, if only one piece is used, it may run the entirety of the rear side or face 702 from the left side edge 604 to the right side edge 606. The purpose of the hook fabric on the rear side or face 702 is to provide removable binding of base 502 to post-operative surgical dressing, for example.

Referring to the illustration of FIG. 7, the left side edge 604 of the rear side or face 702 of base 502 is fixably coupled a piece of hook fabric 704. The piece of hook fabric 704 may be configured into any shape. For instance, hook fabric 704 may have a rectangular, circular, square or other suitable configuration. In the illustration, hook fabric 704 is a rectangular member having a height configured to substantially cover the width of base 502, i.e., from top end 710 to bottom end 712.

The width of hook fabric 704 is configured to provide the desired binding on the left side and for ease of uncoupling from the dressing material. For instance, one edge of the hook fabric starts substantially at the left side edge 604, leaving grip portion 701 between the left side edge 604 and the starting first edge of the hook fabric, and runs towards the right side edge 606 of the base member. Grip portion 701 provides handle for ripping, i.e. uncoupling, the hook fabric from the dressing material.

On the right side edge 606 of the rear side or face 702 of base 502 is fixably coupled a piece of hook fabric 706. The piece of fabric 706 may be configured into any shape. For instance, hook fabric 706 may have a rectangular, circular, square or other suitable configuration. In the illustration, hook fabric 706 is a rectangular member having a height configured to substantially cover the width of base 502, i.e., from top end 710 to bottom end 712.

The width of hook fabric 706 is configured to provide the desired binding on the right side and for ease of uncoupling from the dressing material. For instance, one edge of the hook fabric starts substantially at the right side edge 606, leaving grip portion 703 between the right side edge 606 and the starting first edge of the hook fabric 706, and runs towards the left side edge 604 of the base member. Grip portion 703 provides handle for ripping, i.e. uncoupling, the hook fabric from the dressing material.

Also couplable to base 502 is one or more socks 506. Each of sock 506 is comprised of a transparent netting type fabric 802 and configured to hold a drain reservoir. A transparent sock fabric provides easy view of the reservoir and its content so that the patient would have awareness of the fill level and when the reservoir is ready to be emptied.

The sock 506 is configured with an elastic band at top edge 806 to hold the reservoir bulb in place. In addition, sock 506 is fixably coupled a piece of hook fabric 804. The piece of fabric 804 may be configured into any shape. For instance, hook fabric 804 may have a rectangular, circular, square or other suitable configuration. In the illustration, hook fabric 804 is substantially a rectangular member coupled to one side and close to the top sock 506. Hook fabric 804 provides removable binding of sock 506 to the front side or face 602 of base 502, which is configured as a fabric material having hairy fibers capable of acting as the loop side of a “hook-and-loop” fastener.

Also couplable to base 502 is one or more traps 504. Each of straps 504 comprises a flexible fabric having a top side 902 and a bottom side 904. Top side 902 is configured substantially as the hook side of a “hook-and-loop” fastener and bottom side 904 is configured substantially as the loop side of a “hook-and-loop” fastener. The width and length of strap 504 are configured as necessary to secure extension tube 104 on base 502.

Thus, each flexible strap 504 is configured to provide removable coupling of the extension tube to the front side or face 602 of base 502. When securing an extension tube, the strap is rolled around the extension tube such that its hooks fabric (i.e. 902) is on the outside to provide removable coupling to the front side or face of the base, which comprises loops fabric.

In use, one or more sock 506 and strap 504 are attached to base 502 as illustrated in FIG. 5. Sock 506 and strap 504 may be located anywhere on the front side or face 602 of base 502. Reservoir bulb 102 is placed inside sock 506 and strap 504 is rolled around extension tube 104 (the extension tube may also be rolled-up as shown to reduce overhang) and attached to front side or face 602 of base 502.

Thus, a novel method and apparatus for securing a surgical drain system have been described. It will be understood that the above described arrangements of apparatus and methods are merely illustrative of applications of the principles of this invention and many other embodiments and modifications may be made without departing from the spirit and scope of the invention as defined in the claims.

What is claimed is:

1. An apparatus for securing a surgical drain system comprising:
a base having a front side and a rear side, wherein said rear side is configured to be removably couplable to an object on a patient;
a sock configured to be removably couplable to said front side of said base, wherein inside said sock provides temporary storage for a reservoir of a surgical drainage system; and
a strap configured to be removably couplable to said front side of said base and for securing an extension tube of said surgical drainage system.

2. The apparatus of claim 1, wherein said base is a flexible planar fabric.

3. The apparatus of claim 1, wherein said sock comprises a transparent material.

4. The apparatus of claim 1, wherein said sock comprises a netting material.

5. The apparatus of claim 1, wherein said sock comprises an elastic band for securing said reservoir inside said sock.

6. The apparatus of claim 1, wherein said strap is a flexible material.

7. The apparatus of claim 2, wherein said front side of said base comprises loops fabric of a hook-and-loop fastener.

8. The apparatus of claim 2, wherein said rear side of said base comprises hooks fabric of a hook-and-loop fastener.

9. The apparatus of claim 1, wherein said object on said patient is a post-operative dressing.

10. The apparatus of claim 7, wherein said sock comprises a patch of hooks material for coupling to said front side of said base.

11. The apparatus of claim 7, wherein said strap comprises substantially of hooks material on a first side and loops material on a second side.

12. An apparatus for securing a surgical drain system comprising:
a base having a front side and a rear side, wherein said rear side is configured to be removably couplable to post-operative dressing on a patient;
a sock configured to be removably couplable to said front side of said base, wherein said sock provides secure temporary storage for a reservoir of a surgical drainage system; and
a flexible strap configured to be removably couplable to said front side of said base and for securing an extension tube of said surgical drainage system.

13. The apparatus of claim 12, wherein said base is a flexible planar fabric.

14. The apparatus of claim 12, wherein said sock comprises a transparent material.

15. The apparatus of claim 12, wherein said sock comprises a netting material.

16. The apparatus of claim 12, wherein said sock comprises an elastic band for securing said reservoir inside said sock.

17. The apparatus of claim 13, wherein said front side of said base comprises loops fabric of a hook-and-loop fastener.

18. The apparatus of claim 13, wherein said rear side of said base comprises hooks fabric of a hook-and-loop fastener.

19. The apparatus of claim 17, wherein said sock comprises a patch of hooks material for coupling to said front side of said base.

20. An apparatus for securing a surgical drain system comprising:
a base fabric having a front side and a rear side, wherein said rear side comprises hooks material of a hook-and-loop fastener to provide removable coupling to post-operative dressing on a patient and said front side is configured substantially with a loops material of a hook-and-loop fastener;
a transparent sock comprising a patch of hooks material for removable coupling to said front side of said base and an elastic top for temporarily securing a reservoir of a surgical drainage system inside said sock; and
a flexible strap configured to be removably couplable to said front side of said base and for securing an extension tube of said surgical drainage system, wherein said flexible strap comprises hooks material on a first side and loops material on a second side.
